(19) United States
(12) Patent Application Publication
(10) Pub. No.: US 2017/0157075 A1
(43) Pub. Date: Jun. 8, 2017

(54) Edible Compositions and Methods for Promoting Alpha Brain Waves

(71) Applicant: James Edward Beecham, West Palm Beach, FL (US)

(72) Inventor: James Edward Beecham, West Palm Beach, FL (US)

(21) Appl. No.: 15/055,599

(22) Filed: Feb. 28, 2016

Related U.S. Application Data
(60) Provisional application No. 62/387,836, filed on Jan. 7, 2016.
(60) Provisional application No. 62/386,915, filed on Dec. 16, 2015.
(60) Provisional application No. 62/386,878, filed on Dec. 14, 2015.
(60) Provisional application No. 62/386,785, filed on Dec. 12, 2015.
(60) Provisional application No. 62/386,642, filed on Dec. 7, 2015.

(51) Int. Cl. A61K 31/198; A61K 36/48; A61K 45/06; A61K 31/519; A61K 31/714; A61K 31/675; A61K 31/51; A61K 31/405; A61K 31/685; A23L 33/175; A23L 33/15; A23L 33/105; A23L 2/52; A61J 1/03; A61J 1/05; A61K 9/00

(52) U.S. Cl. CPC A61K 31/198; A61K 9/0095; A61K 36/48; A61K 9/0053; A61K 45/06; A61K 31/519; A61K 31/714; A61K 31/675; A61K 31/51; A61K 31/405; A61K 31/685; A23L 33/175; A23L 33/15; A23L 33/105; A23L 2/52; A61J 1/03; A61J 1/05; A23V 2002/00

(57) ABSTRACT

The present invention provides edible compositions, kits and methods for improving brain function/activity, the composition including at least one alpha brain wave promoting factor including theanine, and at least one neurotransmitter-precursor.

### CROSS-REFERENCE TO RELATED APPLICATIONS

This application claims priority from the following US provisional patent applications: 62/386,915 filed on Dec. 16, 2015, entitled “Edible compositions and methods for promoting alpha-brain waves”, 62/386,878 filed on 14 Dec. 2015, entitled “Alpha brain wave-promoting edible compositions”, 62/386,785 filed on 12 Dec. 2015, entitled “Synergistic promoting of alpha brain waves”, and 62/386,642 filed on 7 Dec. 2015, entitled “Edible compositions for promoting health with safety”. These applications are incorporated herein by reference.

### FIELD OF THE INVENTION

The present invention relates generally to edible compositions for improving mental abilities, and more specifically to methods and compositions for improving mental abilities, brain function and brain activity.

### BACKGROUND OF THE INVENTION

In the industrialized countries, there are millions of people, who suffer from chronic diseases, such as cancer, diabetes, cardiovascular diseases and the like. Additionally, millions of people suffer from anxiety, overwork, under-sleep, chronic fatigue, mental diseases and disorders, Alzheimer's disease, ADD, ADHD, premature aging, obesity and stress-related overweight, psychiatric/psychological disorders which reduce their mental abilities, brain activities, and brain functions. Often, the person or patient receives one or more drugs to improve their mental alertness and/or abilities. Such drugs are often ineffective and subject the user to side effects.

Alternatively or additionally, such people are often chronically stressed. They often consume a diet low in nutrients and high in carbohydrates. They often turn to coffee, caffeinated beverages, sugar and confectionary and weight-increasing high-carbohydrate foods to try to overcome their mental fatigue and/or in response to stress.

Energy drinks and the like are commonly consumed daily by thousands of people. Unfortunately, many such beverages are comprised of vasoactive amines, sugar and caffeine, and are typically highly acidic (in a pH range of 2-5). Some energy drinks have been implicated in lawsuits, citing damage or death to users. The extreme acidity and high caffeine content can cause rapid heartbeat, high blood pressure, dehydration, vomiting, cardiac arrhythmias, seizures, headaches, insomnia, and even death.

Additionally, large quantities of caffeine can cause elevated blood pressure, elevated heart rate and can lead to severe dehydration by causing the body to eliminate water, salt, and nutrients. Common reactions that occur in user/consumers of such drinks include jitteriness, hang-over or crash.

Additionally, animals, including pets and other animals fed by people, suffer from poor diet and related diseases, such as poor mental function and less than optimal brain function/activity, obesity and other conditions related to over-eating by the animal.

Some patent publications in this field include U.S. Pat. Nos. 6,261,589; 6,444,218, 8,197,855, 9,049,879; 4,775,665; 9,198,454; 8,741,319; 9,040,582; 8,187,647; 9,101,580; 9,084,825; 8,852,656; 7,670,619; 6,207,699; 5,958,429; 4,853,377; 4,435,424; 4,377,595; 8,999,424; 6,444,218, 8,197,855, 4,025,613; 5,578,336; 6,294,520; 8,541,359; 8,636,985; 8,852,656; 5,456,677, 8,728,559, 8,148,651, 8,029,819, 8,697,116 and 8,642,097.

There thus remains an unmet need for an edible composition, adapted to effectively promote enhanced mental capabilities and brain functions/activities. There is a further need for such a composition, which is safe and healthy for use, including intermittent or repeated use.

Additionally, there is another unmet need for methods for improving mental abilities, brain function and brain activity in mammalian subjects suffering from mental fatigue, mental stress, anxiety, depression, poor diet and related diseases and disorders, such as stress-induced weight gain and obesity.

Additionally, there is another unmet need for an edible composition adapted to effectively enhance the health of an animal under the care of a person, including pets and other animals.

Additionally, there is an unmet need for promoting the health of an animal, such as in regard to choices of food and other edibles available to be selected by the person responsible for feeding that animal, and wherein that selection of edibles can be improved by providing edibles better adapted to effectively enhance the animal's health, including enhancing in the animal the brain function of satiety and the brain activity of alpha brain waves, and wherein such improvements in edibles, when consumed by the animal, can enable improved animal health, including better weight control in the animal.

### SUMMARY OF THE INVENTION

It is an object of some aspects of the present invention, to provide improved edible compositions for improving brain activity and improving brain function.

The term ‘brain function’, as used herein, comprises generation of a satiety signal or satiety sensation in a mammalian subject's brain. The term ‘brain activity’, as used herein, comprises alpha brain wave activity in a mammalian subject's brain. The term ‘improving brain function/activity’, as used herein, comprises improving, contemporaneously in a mammalian subject, both a brain function and a brain activity.

In some embodiments of the present invention, improved methods and compositions are provided for enabling satiety and enabling alpha brain waves, and thus controlling body weight and countering stress-related deficits.

In other embodiments of the present invention, methods and compositions are provided for improving mental performance, brain activity and brain function.

In further embodiments of the present invention, methods and compositions are provided for reducing deleterious effects of aging, stress, anxiety, depression, poor diet and disease, including reducing overweight and countering stress-related disease such as obesity.

There is thus provided according to an embodiment of the present invention, an edible composition for improving brain function/activity, the composition including;
a) at least one alpha brain wave promoting factor including theanine; and
b) at least one neurotransmitter-precursor including factor comprising a serotonin-precursor.

The term “factor” is used broadly herein to define any element such as a compound, a chemical, a composition, an element, a suspension, a formulation, a moiety or combinations thereof, which comprises, contains, holds or is an active agent.

Additionally, according to an embodiment of the present invention, the composition further comprises at least one choline providing factor.

Additionally, according to an embodiment of the present invention, the composition further includes at least one vitamin.

Additionally, according to an embodiment of the present invention, the composition further includes at least one preservative.

Moreover, according to an embodiment of the present invention, the composition comprises at least one dopamine-precursor (DP) and at least one serotonin-precursor (SP).

Further, according to an embodiment of the present invention, the at least one DP and the at least one SP are in relative metabolic potency balance regarding healthful brain production of serotonin versus contemporaneous healthful brain production of dopamine. This balance will be further discussed below. This balance-promoting feature of the composition is herein termed Balanced Dopamine/Serotonin Production capacity (BDSP).

Yet further, according to an embodiment of the present invention, the dopamine-precursor (DP) is selected from an amino acid, a substantially purified amino acid, an amino acid derivative, a tyrosine, a phenylalanine, L-tyrosine, L-phenylalanine, N-acetyl L-tyrosine, n-methyl-L-tyrosine, and combinations thereof.

Moreover, according to an embodiment of the present invention, the SP is selected from an amino acid, a substantially purified amino acid, an amino acid derivative, a tryptophan, a substantially purified tryptophan, a tryptophan derivative, L-tryptophan, a hydroxytryptophan, a 5-hydroxytryptophan (5-HTP), a plant extract containing 5-HTP and combinations thereof.

Furthermore, according to an embodiment of the present invention, the at least one choline providing factor is selected from a choline, phosphocholine, acetyl choline, phosphatidylcholine, lecithin, citicoline, alpha-glycerophosphocholine (alpha-GPC, L-Alpha glycerylphosphorylcholine, choline alfoscerate), a choline derivative, and combinations thereof.

Additionally, according to an embodiment of the present invention, the edible composition includes at least one of: hydroxytryptophan in amount greater than 15 milligrams per consumed single day dose and less than 1800 milligrams per consumed single day dose, tryptophan in amount less than 6 grams per consumed single day dose, hydroxytryptophan in amount equal to 5% to 35% of total milligrams combined of dopamine-precursor in the composition, one or more tryptophans in total amount 50% to 350% of total milligrams of dopamine precursor in the composition.

Further, according to an embodiment of the present invention, the edible composition is dispensed in at least one of a pill, a lozenge, a tablet, a sublingual, a fizzy effervescent tablet, a capsule, a dry powder, a purified water solution, a suspension, a candy, a beverage, a wafer, a mint, a chocolate candy, a hard candy, a soft candy, a wash, a spray, a sprinkle, a condiment, a common food look-alike, animal food, and combinations thereof.

Additionally, according to an embodiment of the present invention, the edible composition further includes a pharmaceutical aqueous carrier. Furthermore, according to an embodiment of the present invention, the pharmaceutical aqueous carrier includes approximately 50-88 ml water.

Additionally, according to an embodiment of the present invention, the edible composition includes L-theanine, 30 to 800 mg per consumed single day dose, alpha-glycerophosphocholine (alpha-GPC), 10-600 mg per consumed single day dose, 5-hydroxytryptophan (5-HTP), 15-900 mg per consumed single day dose, L-tyrosine, 0-400 mg per consumed single day dose and N-acetyl L-tyrosine, 0-400 mg per consumed single day dose.

Moreover, according to an embodiment of the present invention, the edible composition includes theanine, 30 to 800 mg per consumed single day dose, citicoline, 10-600 mg per consumed single day dose, Griffonia simplicifolia seed extract rich in 5-HTP, comprising 15-1800 mg 5-HTP per consumed single day dose, tyrosine, 20-650 mg per consumed single day dose, and at least one B vitamin.

There is thus provided according to an additional embodiment of the present invention, a method for improving brain function/activity in mammalian subject consuming a nutrient-rich and healthful diet, the method including providing the subject with the edible composition including;
a) at least one alpha brain wave-associated factor including theanine; and
b) at least one neurotransmitter-precursor including factor comprising serotonin-precursor in a pharmaceutically effective amount; thereby improving the brain satiety function and improving the alpha brain wave activity in the mammalian subject.

There is thus also provided according to an additional embodiment of the present invention, a method for improving brain function/activity in a mammalian subject consuming a non-ideal diet, the method including providing the subject with the edible composition including;
a) at least one alpha brain wave promoting factor including theanine;
b) at least one neurotransmitter-precursor including factor for Balanced Dopamine/Serotonin Production capacity (BDSP), in a pharmaceutically effective amount, and
c) at least one choline providing factor; thereby improving the brain function/activity in the mammalian subject.

Additionally, according to an embodiment of the present invention, the method further includes providing the composition in a calibrated container together with instructions for consumption of the edible composition.

Moreover, according to an embodiment of the present invention, the method further includes dividing the composition into volume doses in the calibrated container, for the subject to consume within a predetermined period of time.

Further, according to an embodiment of the present invention, the method further includes instruction facilitating the subject to consume the contents of at least two calibrated containers per day.

Yet further, according to an embodiment of the present invention, the method further includes instruction facilitating the subject to consume the contents of at least three calibrated containers per day.

Additionally, according to an embodiment of the present invention, the method further includes instruction facilitating the subject to consume the contents of at least four calibrated containers per day.

Importantly, according to an embodiment of the present invention, the improved brain activity includes improved alpha brain wave activity in the subject and the contemporaneous improved brain function includes improved satiety enablement.

There is thus provided according to an additional embodiment of the present invention, a kit for improving brain function/activity, the kit including;
a. an edible composition as described herein;
b. a calibrated a container of up to 4 fluid ounces capacity; and
c. instructions for use of the kit, wherein the composition includes sufficient preservative so as to preclude a need for refrigeration of the composition in common use.

Additionally, according to an embodiment of the present invention, the instructions include a divided dose usage schedule for at least a portion of the composition to be consumed in a specified timeframe, such as a sip when hunger is sensed by the mammalian subject or after a meal, or before a meal, or during a meal or hourly in the hours immediately preceding bedtime or the like. Additionally, according to an embodiment of the present invention, the instructions include a divided dose usage schedule for at least a portion of the composition to be consumed a specified number of minutes in relation to time of a meal, such as a third of the composition to be consumed 30 minutes prior to a meal.

Additionally, according to an embodiment of the present invention, the instructions include for at least a portion of the composition to be consumed with contemporaneous consumption of a specified amount of water, such as 1 or 2 glasses of water (8 to 16 fluid ounces of water) to be consumed along with composition or an aliquot of composition.

Additionally, according to an embodiment of the present invention, the instructions include for at least a portion of the composition to be consumed in evening time so as to increase satiety at evening time and thus reduce food consumption prior to bed time, so as to lessen the likelihood of gastric acid damage to esophagus from reflux at night, thus reducing chance of esophageal cancer.

Additionally, according to an embodiment of the present invention, the composition is comprised of at least one neurotransmitter-precursor factor and at least one theanine factor, wherein the composition is configured as an edible composition suitable for consumption by an animal, such as for a pet, said composition useful to control weight of the animal.

### DETAILED DESCRIPTION OF THE EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein. Various documents including, for example, publications and patents, are recited throughout this disclosure. All such documents are hereby incorporated by reference. The citation of any given document is not to be construed as an admission that it is prior art with respect to the present invention.

Herein disclosed is an edible composition which, in the preferred embodiment, when consumed by a mammalian subject in accordance to instructions, benefits mammalian subject's brain function/activity safely, via promoting alpha brain waves and serotonin availability in the mammalian subject's brain, such actions beneficially decreasing anxiety and increasing satiety in the mammalian subject, among other benefits.

The first ingredient of the instant invention composition is a promoter of alpha waves in the brain. Alpha brain waves are neural oscillations in the frequency range of 7.5 to 12.5 hertz, typically arising from synchronous and coherent in-phase constructive electrical activity of neuron cells in mammalian brains, including human brains.

It is known that within the brain, as measured by electroencephalograph (EEG), there are 4 main frequencies of brain waves: alpha, beta, gamma and delta. These tend to occur together, with alpha and beta predominating during waking states, while gamma and delta typically predominate during sleep.

One difficulty, for many people suffering from a disease or mental fatigue or anxiety, is to achieve sufficient alpha brain wave activity, in particular, when the stress in their lives is high. Beta waves typically predominate when working or studying. Yoga can help some people to promote alpha brain wave activity.

Potentially unhealthy ways to increase alpha brain waves include consuming alcohol and smoking marijuana. Meanwhile, engendering a toxic excess of any of the types of brain wave activity is generally considered unhealthy. Those experiencing daily stress or anxiety are likely to benefit from engendering more alpha brain wave activity.

Alpha waves are typically thought to be characteristic of a relaxed mental state. Human subjects in studies have demonstrated alpha brain waves when they were at rest, but alpha brain waves do not necessarily cause tiredness or asleep state. Scientific evidence suggests that alpha brain waves reflect a calm mind. Alpha brain waves appear to be beneficially linked to creativity, problem solving, ease in peak athletic performance, positive mental outlook and certain types of learning enhancement.

A relative and healthy increase in alpha brain waves, especially for stressed individuals, is one beneficial change the instant invention edible compositions seek to engender in mammalian subjects consuming the instant invention edible compositions. Engendering alpha brain waves is one scientifically feasible means to reduce the damage to the body from chronic stress. For example, chronic stress triggers the repeated and ongoing release of a cascade of chemicals, including cortisol.

One action of cortisol is to signal the body to replenish its food supply. When cortisol is chronically elevated, this can lead to over-eating which over-eating can cause an increase in body fat, especially “visceral fat” deep in the abdomen and surrounding organs. Such visceral fat is not only difficult to subsequently get rid of, but it also is known to chronically release chemical triggers for inflammation. Chronic inflammation in the mammalian body increases the likelihood of developing heart disease, cancer or diabetes, as well as many other diseases.

The instant invention edible compositions comprise multiple factors. For example, a composition comprises a) at least one alpha brain wave promoting factor, b) at least one neurotransmitter-precursor, and optionally c) at least one choline containing factor, and optionally d) at least one preservative.

In one embodiment of the instant invention, the alpha brain wave promoting factor comprises theanine, (CAS Number 3081-61-6). Research (Nobre, 2008) identifies theanine as capable of promoting alpha brain wave activity when consumed. Theanine is also termed “an alpha brain wave-associated factor” herein.

Theanine (gamma-glutamylethylamide) is an analog of glutamate and glutamine. Theanine is a predominant amino acid derived from green tea, the main component responsible for the taste of green tea (Camellia sinensis). L-theanine (the levo enantiomer) is capable of crossing the blood-brain barrier. L-theanine is a form of L-glutamic acid, able to help increase and modulate alpha brain waves, which waves are believed to release serotonin in the brain, and through such engendered alpha brain waves help promote a feeling of calm, relaxed alertness (Head, 2009).

Some preferred embodiments comprise choline. According to other embodiments, the choline containing factor is selected from choline, phosphatidylcholine, acetylcholine, alpha-glycerophosphocholine (alpha-GPC), lecithin, citicoline, a choline derivative, and combinations thereof.

Choline is an essential nutrient for brain health. The recommended adequate intake of choline, according to Oregon State University's Lines Pauling Institute, is 425 milligrams choline per day for women and 550 milligrams choline per day for men. Scientific evidence suggests choline is important for intelligence and synaptic plasticity. Synaptic plasticity is the healthy ability of synapses, the neurochemical connections between neurons, to strengthen or weaken over time, in response to increases or decreases in their activity.

Choline and choline derivatives are used within the brain, among other uses, as substrate for formation of phosphatidylcholine, a needed ingredient in healthy neuron cell membranes. Neuron cell membranes are key in the mechanism by which neurons generate alpha brain waves, as will be further discussed below. Choline is also important to brain health as a precursor to acetylcholine, a neurotransmitter in the brain which is important for memory.

Mammals typically derive choline from certain foods in diet, however an estimated 90% of the human population consumes less than the recommended amount of choline per day. A choline deficiency can greatly impair memory and reasoning functions, while also making it harder to focus. Choline deficiency may also negatively affect mood. Among the embodiments herein disclosed are embodiments of the instant invention suitable for those mammals consuming a diet suitably rich in nutrients, such as having adequate choline consumption and adequate consumption of protein. Some other embodiments are more suitable for those whose diet comprises relatively inadequate choline consumption and relatively inadequate consumption of protein.

In a preferred embodiment, the choline-comprising factor comprises alpha-glycerophosphocholine (alpha-GPC). In another preferred embodiment, the choline factor comprises citicoline. These forms of choline are known to cross the blood-brain barrier readily from the blood, thus providing choline directly to the brain for use, among other functions/activities, in the production in the brain of acetylcholine and phosphatidylcholine (Kidd, 2005).

Acetylcholine levels tend to decrease within the aging mammalian brain. Scientific evidence suggests maintaining a healthful level of acetylcholine within the human brain may forestall decline in memory function. Reduced memory function is often seen in aged humans.

The benefits of providing a choline containing factor within some of the instant invention edible compositions include improving the integrity of neuron cell membranes for the mammalian subject/consumer of the said instant invention compositions, especially for those mammalian subjects/consumers whose diet is deficient in choline. Integrity of neuron cell membranes is one key factor which enables robust alpha brain wave formation, as will be further discussed below.

Preferred embodiments of the compositions of the present invention comprise at least one neurotransmitter-precursor factor. In some embodiments, the at least one neurotransmitter-precursor comprises at least one precursor of brain serotonin. In other embodiments the at least one neurotransmitter-precursor factor comprises at least one precursor of brain serotonin and at least one precursor of brain dopamine.

Some preferred embodiments of the instant invention edible compositions comprise a balanced set of precursors/factors related to production in mammalian subject/consumer's brain of the neurotransmitters dopamine and serotonin; balanced in the sense that the instant invention composition acts metabolically within the mammalian subject's brain to assist in forestalling depletion of dopamine in the brain of the mammalian subject, despite the fact that the mammalian subject's brain is also contemporaneously enzymatically metabolizing the serotonin precursor(s) from the composition into serotonin. The mechanism whereby this balance occurs will be further described below.

Dopamine depletion syndrome (DD), as will also be further discussed below, is known to occur when an inadequate diet is consumed, such as a diet low in protein, and/or when a mammalian subject consumes excess amounts of serotonin-precursor. In the former case there is insufficient substrate supply of dopamine-precursor, while in the latter case, the enzyme aromatic amino acid decarboxylase (AAAD) within the brain is occupied by serotonin-precursor conversion to serotonin, and so occupied to the detriment of dopamine-precursor L-dopa conversion in the brain to dopamine.

One common factor in these two pathways to DD is inadequate conversion of dopamine-precursor in the brain into dopamine. A preferred embodiment of the instant invention edible composition comprises dopamine-precursor as an additional supply to the mammalian subject's brain as a means to provide substrate for AAAD to produce dopamine. Meanwhile, the instant invention edible compositions, applied per provided instruction for use, avoid spikes in over-supply of serotonin-precursor so as to avoid or reduce the chance for the second above described pathway to DD to occur, i.e. wherein the enzyme aromatic amino acid decarboxylase (AAAD) within the brain is occupied by serotonin-precursor conversion to serotonin, and so occupied to the detriment of dopamine-precursor L-dopa conversion in the brain to dopamine.

Dopamine, a brain neurotransmitter, does not cross the blood brain barrier (BBB). As a result, any dopamine which is available to the mammalian brain must be produced within the brain itself. This production of dopamine in the brain is dependent upon enzymes and substrate-precursors. The enzymes which would participate in producing dopamine within the brain, from dopamine-precursors, are also the same family of enzymes which may act, within the brain, to produce serotonin from serotonin-precursors. Thus, as described above, the enzyme activity can be withdrawn from dopamine production when excess serotonin-precursor is presented to the mammalian brain. This can cause low dopamine levels within the brain. Preferred embodiments of the instant invention, as described above, beneficially restrict the amount of serotonin-precursor delivered over time to the mammalian brain.

Low dopamine levels in the brain are well known to occur as a side effect of certain addictions, as well as known to occur with certain medical conditions such as schizophrenia and neurodegenerative diseases, such as Parkinson's disease. Another, less well-known cause for depleted dopamine in the brain is chronic consumption of excess serotonin precursors as supplements. When excess serotonin-precursor compositions are consumed, especially when the mammalian subject/consumer so consumes chronically, while also consuming a diet low in protein, then dopamine depletion is even more likely to occur.

For example, studies have shown that high doses of 5-hydroxytryptophan (5-HTP), a potent serotonin-precursor, can lead over time to depletion of brain dopamine (Hinz et al., (2011); Hinz et al., (2012)). One common source for 5-HTP is from an extract of seeds of the woody climbing shrub, Griffonia Simplicifolia, a native plant of West and Central Africa. Mechanisms by which dopamine depletion in the brain can occur include enzyme activity being diverted within the brain for near exclusive use in converting the final step serotonin-precursor 5-HTP into brain serotonin. Such diversion deprives the final step dopamine-precursor L-dopa of the opportunity to be sufficiently metabolized into dopamine in the brain. This situation occurs because the same enzyme, L-aromatic amino acid decarboxylase (AAAD), is responsible for both conversions in the brain.

Therefore, where neurotransmitter-precursor-unbalanced supplements, i.e. with serotonin-precursor in large amount and little if any dopamine-precursor, are taken by a mammalian subject/consumer on a regular daily basis, symptoms of dopamine depletion can arise. DD can be caused/exacerbated in those consuming a nutrient-poor diet. This is because protein in diet can be a source of amino acid to the brain, and amino acid tyrosine and/or amino acid phenylalanine, typically derived from protein-rich food digestion, are known dopamine-precursors. Thus, a diet low in protein will contribute to low levels of dopamine-precursors in the brain.

Symptoms of dopamine depletion can include depression, anxiety, fatigue, attention deficit, blunted affect, confusion, low libido, confused thinking, social withdrawal, balance difficulties, tremors, babbling speech, disorganization and apathy. The instant invention edible compositions which optionally contain a dopamine-precursor factor such as tyrosine or phenylalanine, have, as a benefit to the nutrient-deficient mammalian subject/consumer, the addition of dopamine-precursor which may serve to confer on the mammalian subject a relative resistance to dopamine depletion syndrome.

Such compositions are herein referred to as balanced. The balance feature of such a composition is herein referred to as Balanced Dopamine/Serotonin Production capacity or BDSP. In a preferred embodiment, the BDSP set of factors within the edible composition comprises at least one dopamine precursor, for example amino acid tyrosine and/or amino acid phenylalanine, or their derivatives, and at least one form of serotonin-precursor. One or more varieties of tryptophan are typically utilized within the instant invention edible compositions as serotonin-precursor.

The potential for serotonin production is understood to vary, on a milligram to milligram basis, from one tryptophan moiety or derivative to another. Thus the said balanced instant invention edible compositions are configured so as to tend to maintain the said balance by virtue of adjusting, within the individual composition, the amounts of the dopamine-precursor(s), in consideration of their individual and combined potency in yielding dopamine in the brain in comparison to the amounts of serotonin-precursor(s), in consideration of their individual and combined potency in yielding serotonin in the brain.

Another factor, that of avoiding an excess of serotonin production in the brain, is also part of the inventive steps herein, as will be further discussed below. The assessment for purposes of selecting milligram amounts of factors in at least some edible compositions of the instant invention includes assessment of activity of one or more of the composition ingredient's ability to be absorbed after oral ingestion, to circulate in the mammalian subject's/consumer's blood, and to cross the blood-brain barrier (BBB), thus entering the mammalian subject's brain. Each neurotransmitter-precursor has its own characteristics in these regards.

For example, tryptophan, as an amino acid circulating within the blood, will be subject to competition at the amino acid transporter of the BBB from at least some of the other amino acids in the blood. Meanwhile, 5-hydroxytryptophan (5-HTP) is thought not to be subject to as intense a competition from such circulating amino acids in the blood, by virtue of 5-HTP being already in a form relatively more easily cleared through the BBB.

One practical result of such a difference in bioavailability of various serotonin-precursor factors is that compositions under the instant invention which are dependent largely on tryptophan as a serotonin precursor, rather than on 5-HTP, become relatively more effective in their supplying brain serotonin production capacity when such composition is consumed at a time when the concentration of competing amino acids is relatively lower in the blood. Typically, this situation occurs in-between meals, especially in the time just before the next meal.

Meanwhile, compositions dependent solely or largely on 5-HTP as serotonin-precursor are less altered in absorption efficiency, or in achieving relatively high levels of 5-HTP in circulating blood and in the brain, even if taken with meals or shortly after meals or snacks. These facts are utilized in formulating of compositions of the instant invention, as well as in drafting instructions for use of embodiments of the instant invention edible compositions, as will be further discussed below.

Furthermore, as an inventive step herein, the instructions for use of compositions of the instant invention are optionally adjusted to take into account the body weight of the mammalian subject/consumer. For example, larger mammals typically need higher doses of factors to achieve benefits herein disclosed, as compared to smaller body weight mammals.

With regard to the serotonin precursor 5-hydroxytryptophan (5-HTP, CAS No.: 56-69-9), this molecule has an excellent characteristic of absorption after oral ingestion and also is adapted to readily penetrate through the blood-brain barrier. It has been estimated that 70% of an oral dose of 5-HTP ends up producing serotonin in the mammalian subject's body, with a substantial amount of serotonin thus produced in the mammalian subject's brain.

Thus 5-HTP has relatively more potential or potency, on a milligram by milligram basis, to yield production of serotonin in the brain, as compared, for example, to L-tryptophan or other tryptophan moieties. With 5-HTP in the composition, starting with the milligram amount of the 5-HTP ingredient, the balancing amount of dopamine-precursor calculated to meet or approach the goal of achieving said balance, i.e. at least minimal healthy enzyme activity devoted to dopamine production in the brain so as to avoid dopamine depletion, is approximately 5 to 10 times higher than would be the case if the composition had the same milligram amount of the less potent L-tryptophan instead of 5-HTP. In other words, on a milligram to milligram basis, a milligram of 5-HTP requires more dopamine-precursor to balance the 5-HTP versus the lesser amount of milligrams of dopamine-precursor needed to balance a milligram of L-tryptophan in a composition.

Just as low dopamine can be detrimental to brain health, so also serotonin excess can cause health problems. Generally speaking, beneficial levels of serotonin are associated with feelings of happiness and relaxation. Low serotonin levels, on the other hand, are linked to weakened immune function and depression. An excess of brain serotonin can cause a condition known as serotonin syndrome (SS). SS can lead to excessive nerve cell activity and symptoms such as confusion, agitation, restlessness, dilated pupils, headache, changes in blood pressure or temperature, nausea, vomiting, rapid heart rate, tremor, loss of coordination, muscle twitching, shivering, ‘goose’ bumps, heavy sweating, seizures, unconsciousness, and in extreme cases, death.

At least some preferred embodiments of the instant invention's edible compositions accordingly are comprised of factors such that BDSP is enabled while SS risk is minimized. In other words, such embodiments, as suitable, for example, for those mammalian subjects consuming a non-ideal diet, are herein termed ‘balanced’ by virtue of being comprised of factors containing appropriate amounts and moieties of dopamine-precursor and also containing such serotonin-precursor moieties and amounts whereby excess brain serotonin secondary to such edible composition's consumption per provided instruction is avoided or rendered very unlikely.

Furthermore, among the instructions of use is optionally the advice for consumers of SSRI medications to avoid consuming the instant invention compositions, so as to avoid excess serotonin activity in the brain. Furthermore, it is recognized that the level of general nutrition in the mammalian subject can affect the level of brain enzyme AAAD available. For example, adequate vitamin D is believed to be a contributing factor for production of adequate AAAD in the human brain. Vitamin B6, niacin, and magnesium also help convert 5-HTP to serotonin. Hence, it is advisable to maintain a healthful diet, and instructions optionally include such advice.

This safety factor of limiting the maximum amounts permissible of serotonin-precursor within the instant invention embodiments assists in fulfilling the goal of the instant invention edible compositions to provide improvement in brain function/activity which avoiding SS in the mammalian subject. For the instant invention edible compositions, this goal is further served by instructions/dosage schedules as herein disclosed, and by selecting maximum amount limits in total and for each type of serotonin-precursor used in formulating said compositions, which maximum limits are selected in reference to results of scientific studies.

As example of serotonin-precursor studies in mammalian subjects/humans, studies in which the tryptophan moieties were administered to human subjects over a time period of weeks to months, are especially useful guides in setting maximum limits for daily dosage. 5-HTP-containing factors may have a racemic mixture of dextro- and levo-forms (DL-5-HTP). In some studies reported in the scientific literature, racemic 5-HTP use is apparently sometimes referred to as 5-HTP or as the levo enantiomer L-5-HTP, apparently reflecting the fact that the levo form of 5-HTP is the active form in regard to neurochemical and behavioral effects (Penn 1977).

Examples of 5-HTP use in scientific studies include a controlled trial, at a dose of 300 mg per day, in which 5-HTP was shown to be effective in reducing many symptoms of fibromyalgia, including pain, morning stiffness, sleep disturbances, and anxiety. Migraine attacks were reduced in frequency, severity, and duration in 90% of those taking 400 mg per day of 5-HTP in a well-controlled trial. Larger doses of 5-HTP (600 mg per day) were found to be as effective as prescription medications for reducing migraine headache attacks in adults in two double blind studies.

Children who suffered from migraines and had problems sleeping responded well to a daily dose of 5-HTP equal to 20 mg for every 10 pounds of body weight in a controlled study. For depression, 300 mg per day is often effective. A single 100 mg nighttime dose of 5-HTP was sufficient to improve the duration and depth of sleep in one placebo-controlled study. Appetite reduction and weight loss (averaging 11 lbs weight loss in twelve weeks) has occurred with doses of 5-HTP in the range of 600-900 mg daily. Side effects or interactions reported during these studies mainly consisted of mild gastrointestinal disturbances in some people, mild nausea, heartburn, flatulence, feelings of fullness, and rumbling sensations.

It is understood that a common source of 5-HTP supplement is from seeds of the Griffonia simplicifolia plant. Such 5-HTP-rich extracts may vary in percent content of 5-HTP, and hence the relative serotonin-production capability strength of any particular 5-HTP seed extract composition may vary from that of another. Using the above data as guide, and considering, as an example, the circumstance where 5-HTP is the sole serotonin-precursor in a particular edible composition under the instant invention, the single-day dose total of 5-HTP is envisioned, for human subjects, to be in a range from 30 mg 5-HTP per day up to, generally speaking, approximately 900 mg to 1800 mg of 5-HTP maximum per single day dose.

Meanwhile, for the dopamine-precursors in certain embodiments of the instant invention edible compositions, the efficacy of BBB transit of such dopamine-precursors, for example phenylalanine or tyrosine, is known to be adversely affected by the presence of a high titer of other amino acids in the blood, as previously mentioned. Hence, optimum efficacy for the instant invention edible compositions containing dopamine-precursors obtains, in general, when the said composition is consumed on a relatively empty stomach, for example prior to a meal or snack. At this time of empty stomach conditions in the mammalian subject, absorption of said dopamine-precursors would likely be better due to the fact that competing amino acid titers are relatively lower in the intestine, and due to the fact that interfering food in the digestive tract would be less. Thus, when the dopamine-precursors are so consumed, they yield higher levels in blood of these specific dopamine-precursor amino acids, allowing them to outcompete other blood amino acids for transport into the brain via the amino acid transport mechanism at the BBB. Instructions for use may accordingly vary depending on composition formulation.

It is understood that a person may manage/provide his own edibles for his/her own consumption. A person may manage/provide at least some of the edibles for consumption by another mammalian subject, either a person or an animal. Such a person is herein termed a person responsible (PR). For example, a pet owner is typically the PR for his/her pet. At least one parent is typically the PR for a child for whom that parent provides edibles for the child to consume. A care-giver for a parent is typically the PR for at least some of the edibles consumed by that parent. It is, generally speaking, to such a PR that the instructions for use of the edible compositions, as herein disclosed and described, are particularly useful. Following such instructions is typically useful in enabling the beneficial amounts of, and beneficial timing for the mammalian subject's consumption of the edible compositions disclosed herein.

In a preferred embodiment of the instant invention, the edible composition is in the form of animal food, such as pet food. In another preferred embodiment, the composition is in the form of a beverage, for example suitable for drinking by either a human or a pet. Instructions optionally direct PR to administer sips of such beverage, for example to administer a sip aliquot to the mammalian subject upon first feeling hunger, or in the case of a caregiver the first noticing actions which demonstrate hunger.

It is understood that, in general, a larger mammalian subject, having greater body weight, may require a larger dose of edible composition to receive the same satiety signal advantage as a lower body weight individual would receive from a smaller dose of composition. Optionally, instructions contain information which provides PR data useful to adjust the amount of composition to administer in relation to mammalian subject/consumer's body weight.

Another option in instructions is to instruct a PR to administer an aliquot of a composition, wait a specified period of time, such as 2 hours, and then administer another aliquot, and so on. It is understood that the PR, in regard to his/her own consumption of edibles, will be administering these aliquots to himself/herself in compliance to such instructions.

In this manner, at least some of the beverage is likely to be consumed when the mammalian subject's stomach is relatively empty, thus improving absorption for at least some of the factors. In this manner, the level of enzyme AAAD required to metabolize the relevant ingredients is more likely to be available and adequate within the brain, given that the gradual application of the factors within the said composition administered in aliquots over time tends to place less demand over time on the enzymes serving to convert tryptophan in the brain into serotonin, as compared to administration of the full amount at one time. The divided dose schedule also enables AAAD enzyme activity to be more available to contemporaneously convert dopamine-precursor in the brain into dopamine, whether such dopamine-precursor is provided from healthful diet, or by the consumed composition.

It is further understood that, within the body, L-tryptophan can undergo conversion into 5-hydroxytryptophan. From that point, the said 5-HTP behaves, within the body, in the same manner as any other molecule of 5-HTP.

Tryptophan is an essential amino acid, meaning the body needs tryptophan but cannot produce it. Hence, unless supplemented, tryptophan must be obtained from diet, and thus the amount of tryptophan-rich foods consumed is also a factor. Tryptophan-rich foods include nuts, seeds, tofu, cheese, red meat, chicken, turkey, fish, oats, beans, lentils, and eggs.

A previous concern about consumption of tryptophan supplements was eosinophilia myalgia syndrome (EMS), a potentially serious but rarely encountered side effect, the precise cause of which is still unclear. Some scientists believe a cluster of 1500 EMS cases in the late 1990's was caused by contaminants in a manufacturing process which used genetically engineered bacteria to produce 5-HTP as an OTC supplement. Following the elimination of that Japanese manufacturer's product from the market, cases of EMS virtually disappeared and the FDA reinstated tryptophan as “Generally Regarded as Safe” (GRAS).

In 2011, Allen et al., reported a single EMS case in an individual who, while supplementing with several non-tryptophan supplement products, was supplementing also with tryptophan (Allen et. al. Arthritis Rheum. 2011 November; 63:11). The report reviewed data from the adverse reactions database representing the 10 years which followed the removal of the Japanese manufacturer's product from the market. The authors of the study were able to identify only a possible 4 other EMS cases in the data. However, as they pointed out, even these ‘possible’ cases of EMS were complicated by the fact that each of those 4 individuals possibly suffering from EMS secondary to tryptophan supplementation had also been taking other supplements, not just tryptophan. The authors further pointed out that EMS has also been reported in individuals who had never taken tryptophan supplement, such as one individual who apparently got EMS from eating cashew nuts.

In summary, as of the time of this writing, it appears that current reasonable scientific advice regarding tryptophan supplements, for individuals who choose to supplement tryptophan, is that EMS risk from tryptophan supplementation is vanishingly small, if it still truly occurs. Of course, it remains advisable to recommend to any consumer of supplements that, if symptoms of allergy or muscle pain or other adverse reaction should develop, they should stop all supplements and seek medical advice.

Tryptophan/5-HTP supplementation may be problematic in persons taking SSRI medications (Selective serotonin reuptake inhibitors or serotonin-specific reuptake inhibitors) or other anti-depressants/psychoactive prescription drugs such as monoamine oxidase inhibitors (MAOI's). Individuals on SSRI medications are wisely advised not to supplement tryptophan or tryptophan derivatives such as 5-HTP, due to the potential for excess serotonin to arise within the brain in the situation of consuming SSRI medication while also consuming such tryptophan supplements.

Another issue sometimes raised regarding possible side effects of supplementing tryptophan/5-HTP is cardiac valvular problems, in view of the fact that some patients with carcinoid tumors which are found to be producing serotonin are known to develop cardiac valvular complications. However, high serotonin diets have been examined in studies reported in the scientific literature, with the conclusion that elevations of serum serotonin from such diets have not been consistent or on the order of that seen in carcinoid tumor patients (Hoshino 1979). Furthermore, Examine.com, an online authority regarding scientific reports on use of supplements, summarizes data in humans regarding 5-HTP supplementation as follows (accessed 5 Jan. 2016): “No toxicity reports have been noted with doses (of 5-HTP) at or below 50 mg/kg bodyweight”

Regarding the inventive step to select a milligram amount of serotonin-precursor within the instant invention edible compositions, the above facts are taken into account. Furthermore, as earlier mentioned, the type of tryptophan moiety in the composition matters to the absorption, distribution in the body and potency of serotonin production in the brain. The oral ingestion of a milligram of L-tryptophan as serotonin-precursor is viewed, in general, and as earlier mentioned herein, as only approximately ⅕ to 1/10 as potent on a milligram to milligram basis regarding serotonin production in the brain as oral ingestion of a milligram of 5-HTP. Accordingly, for compositions of the instant invention containing, for example, 100 mg of tryptophan as the sole serotonin-precursor in the composition, the choice of an appropriate amount of dopamine-precursor in milligrams for balancing that composition would be lower as compared to a higher milligram amount of dopamine-precursor needed for balance in a composition containing 100 mg of 5-HTP as the sole serotonin-precursor.

In order to further disclose and clarify the method of compiling of the edible compositions of the instant invention, including those edible compositions having or lacking dopamine-precursor and those compositions having or lacking choline, and those edible compositions containing dopamine-precursor or containing choline or both, the following formulas are disclosed as examples, beginning two composition containing dopamine-precursor and choline, followed by a composition containing neither choline nor dopamine-precursor:

Composition 1, which contains only 5-HTP as serotonin-precursor and only L-tyrosine and L-phenylalanine as dopamine-precursors: Amount of 5-HTP in milligrams=5% to 35% of total milligrams combined of L-tyrosine plus L-phenylalanine and wherein 5-HTP is in range of 15 mg to 900 mg per day.

Composition 2, which contains only L-tryptophan as serotonin-precursor and only L-tyrosine as dopamine-precursor: Amount of L-tryptophan in milligrams=50% to 350% of total milligrams of L-tyrosine and provided L-tryptophan does not exceed approximately 3 grams/day.*

*The L-tryptophan daily safe limit is based, at least in part, on results of a double-blind, randomized, controlled clinical trial (Steinberg et al., 1999) in which 37 patients with premenstrual dysphoric disorder were treated with 6 grams L-tryptophan daily for 17 days from the time of ovulation to the third day of menstruation, during three consecutive cycles. In addition, 34 patients were given a placebo. Compared to the placebo group, the women receiving the L-tryptophan experienced significant (p=0.004) therapeutic effect for the cluster of mood symptoms comprising the items dysphoria, mood swings, tension and irritability. Notably, of 71 women in the study, only 1 withdrew due to drug side effects.

For compositions containing neither dopamine-precursor nor choline, the following is disclosed as example:

Composition 3, suitable for a mammalian subject consuming a nutrient-rich diet, comprises a single-day dose comprising serotonin precursor 5-HTP within range of from 30 mg to 1800 mg 5-HTP and theanine in amount within range of from 30 mg to 1800 mg. The higher amount ranges, it is noteworthy, are envisioned to be useful, for example, in persons having an extremely large body weight or for a large animal for which satiety enhancement is also desired.

The discussion herein recognizes that there are many supplements on the market, including some not conforming to the safety features of the compositions for the instant invention. Some such supplements, if consumed, may subject the brain of the mammalian subject/consumer to excess serotonin-precursor. Mammals so consuming may experience symptoms of serotonin syndrome or dopamine depletion or both. Dopamine depletion can result in symptoms such as depression, chronic boredom, apathy, chronic fatigue and low physical energy. Low protein diet makes dopamine depletion even more likely. This is because a low protein diet reduces the supply of amino acids from digestion of dietary protein which typically serve as dopamine-precursor substrate in the brain.

For individuals with symptoms of dopamine depletion due to consumption of such compositions, their doctor may not always realize the cause of their symptoms. In that situation, the doctor may prescribe neurological drugs to deal with such symptoms. Neurological drugs are well known to have potentially debilitating side effects. Thus, by consuming the instant invention edible compositions in accordance with instructions, rather than consuming such supplements which may promote SS and/or DD, mammalian subjects are better enabled to avoid not only DD and/or SS, but also to avoid the side effects of psychoactive drugs which might otherwise have been prescribed to them.

A feature of the activity of at least some of the instant invention edible compositions, when consumed, can be said to be the synergistic nature of the benefits of the ingredients in their action within the mammalian subject/consumer's brain. Whether a mammalian subject consumes a composition of the instant invention which has relatively less or no dopamine-precursor/choline, as for example may be suitable for a mammalian subject consuming a healthful diet, or whether a mammalian subject whose diet has a deficiency in the supply of dietary protein and/or dietary choline, and for whom a composition of the instant invention comprising dopamine-precursor and choline would be appropriate, it is useful to understand aspects of the alpha brain waves enabled by consumption of either of these two types of edible compositions of the instant invention.

Promotion of alpha brain wave activity is derived from action of the alpha wave promoter of the composition, in conjunction with the action of the neurotransmitter-precursor(s) in the composition. Choline, if present in adequate amount from diet and/ro from the composition, also has a role. Alpha brain wave activity occurs to boost calm focused thought via synchronized actions of neurons in networks. Neurons each utilize neurotransmitters to transmit their signals across synapses from one neuron to the next in the network. During alpha waves, the signal transmission of neurons in a network in the brain occurs in a synchronized manner. This alpha wave generation utilizes neurotransmitters across the synapses, and operates via intact cell membranes of neurons. The wave sequence occurs via neurons maintaining, then timely reducing, the polarity along their respective neuron cell membranes, as further discussed below.

The alpha wave discharges occur, in synchrony, enabled by membrane depolarizations occurring after network neurons collectively and individually develop sufficient electrical potential across these membranes. This electrical potential is typically achieved by a neuron accumulating negative ions such as chloride at the interior of the neuron's cell membrane, the exterior then being more positive in charge, and resisting transmission of a signal. As the cell membrane depolarizes in turn within a neuron of the network, this transmits a signal towards the synapse of that neuron. This depolarization carries the signal towards the synapse occurs. At the synapse, a space between neurons, cannot transmit the signal electrically across the space, so the network neurons use chemicals, i.e. the neurons release neurotransmitters, which chemicals carry the signal across the synapse space to the next neuron in the network, and so on.

The ability to carry out this activity thus depends on the proper amounts and function of neurotransmitters and on the capability of the neuron cell membranes to maintain polarity, then depolarize in sequence as needed, then quickly re-polarize the membrane in sequence. The coordinated cycles of membrane polarization, depolarization, and then re-polarization of the neurons in the network in sequence, at the frequency of the alpha wave, appears to be key to the benefits alpha waves provide. This activity thus depends on multiple capabilities, including maintaining the structural integrity of the cell membranes of the neurons. Choline contributes membrane structural integrity, and thus to alpha wave capability.

In other words, neuron membrane integrity, as maintained by membrane components such as phosphatidylcholine, enables alpha brain waves, by supporting the polarization/depolarization/repolarization capability and activity. Serotonin, known to be released in increased amount during alpha brain wave activity, apparently also enables the activity. Supplying adequate serotonin and choline to the brain therefore appears to synergistically enable alpha brain waves. Choline provides substrate for phosphatidylcholine, a key structural component of healthy neuron membranes. Theanine is a well-documented promoter of alpha brain waves. Furthermore, aspects of neuron signaling and memory depend upon acetylcholine in the brain, a neurotransmitter derived from choline.

The above disclosed interlinkage between physiology and the factors present in the compositions of the instant invention helps to explain how the ingredients of the instant invention edible compositions operate to provide benefits to a mammalian subject/consumer with regard to mental function, brain function and brain activity. For example, satiety is enhanced when the instant invention edible compositions are properly utilized. Studies carried out by the instant inventor document such enablement of satiety, as described below.

Controlled studies on an adult male human subject were carried out using 2 different beverages comprising edible compositions prepared in accordance with the instant invention. The studies utilized a control period of 5 days wherein the human subject sipped water instead of a composition, such sips when hunger first was sensed, and hourly for the 3 hours immediately preceding going to bed at night. The subject reported no significant reduction of hunger during the control period, and food consumption was unchanged. By contrast, during the two 5 day periods of consuming similarly timed sips of each of the 2 beverage compositions, composition A and composition B, the subject reported an easily noticed and significant reduction in hunger, typically occurring beginning approximately 15-30 minutes after such sips were consumed and lasting for 1 to 2 hours.

As a result of this reduced hunger, the subject, during the two 5 day periods of administration of the 2 beverage compositions, comfortably was enabled to consume significantly less food as compared to food consumption amounts during the control period. This difference in food consumption was especially evident in regard to fatty food and carbohydrate-containing food, while protein consumption tended to remain the same. The amount of reduction varied day to day from approximately 25% reduction to 50% reduction, with the greater reduction during the first days of use of composition A.

A significant difference was also noted in regard to body weight measurements. The subject was weighed daily during the control phase and during the two test phases. The subject's body weight during the control phase did not change significantly. Subject's body weight during the test phase of composition A and during the test phase of composition B demonstrated a gradual decrease. By the end of test period of composition A, the subject had lost 2 pounds body weight as compared to zero weight loss during the control phase. A further 1.5 pound decrease in subject's body weight was documented after 5 days of consumption of composition B.

The subject further reported a slight but progressive increase in looseness of clothes in his waist area when near the end of the second test phase. The subject also, when questioned, reported mild increases in feelings of calm and sociability, which he reported varied in intensity over the test phases without euphoria or disorientation or other undesirable effect. The subject reported he was able to carry on daily activities in a normal manner during the test phases. The subject also reported a significant increase, during test phase, of his readiness and desire to exercise, such readiness translating into his undertaking an increased number of walks of 1 to 2 miles during the test phase as compared to control phase.

The human subject reported that during the test phases of consumption of both composition A and composition B, he experienced an increased ability to avoid eating food in the 3 hours prior to bedtime. This ability varied in intensity from day to day. The human subject reported a significant increase in the number of nights he went to bed with a relatively empty stomach during the test phases (4 days out of 5 with composition A and 3 days out of 5 with composition B), as compared to the control phase wherein the number of times he went to bed with a relatively empty stomach (1 night out of 5) was significantly less.

Although the subject was not tested for memory, the compositions of the instant invention may have applicability for improving memory. It is estimated that up to 90% of people have less than optimal consumption of choline. But choline, such as is present in some of the compositions of the instant invention may assist with memory. This is because acetylcholine production, derived from choline, is one factor which enables memory. It is known, for example, that acetylcholine levels tend to decrease within the aging brain. Declining memory function is common in the older population. Scientific evidence suggests maintaining a healthful acetylcholine level within the brain may forestall age-related decline in memory function. Also, the aging brain often demonstrates dysfunction in cell membrane integrity, as well as reduced serotonin and/or dopamine content.

Within some of the compositions, a purified water solution is used. Such solutions, when consumed, can provide quick absorbability for the ingredients into the mammalian subject. Furthermore, an edible composition configured as a purified water solution comports with the habit of many people generally to drink a beverage in the morning, such as a cup of coffee or an energy drink. Thus the instant invention, in a preferred embodiment, is envisioned to be dispensed as a purified water solution, conducive in form to being accepted and selected readily by people on a regular basis.

The human subject consuming compositions A and B, as described above, reported the taste of those beverages was similar to sweet apple juice, apparently from the combination taste of the malic acid of the compositions and the sucralose. These compositions did not contain a specific apple-flavor ingredient.

For determination of container size, the instant inventive steps for beverage compositions, take into consideration that a minimum size which allow ingredients/factors to maintain solution is useful, as well as a container size and manner of container presentation which will best enable a wide variety of people to readily adopt use of the instant invention edible compositions on a regular basis. In regard to the fluid ounce capacity of container for purified water solutions of the instant invention, the theanine ingredient typically requires a minimum of approximately 100 milligrams per dose to average size human to produce satisfactory alpha brain wave effect. The serotonin-precursor ingredient must also be dissolved. For those compositions containing a choline ingredient and a dopamine-precursor ingredient, further water is needed to support dissolving of these factors. Together, all factors in milligrams within a preferred embodiment of the instant invention typically total approximately within the range 0.6 to 3.3 grams for single-day dose.

The size container having a sufficient size to maintain this amount of solute in solution and simultaneously of size to be adopted readily by people for use is judged by an inventive step, to be a container approximating 2 to 3 fluid ounce capacity or thereabouts for a single-day dose. Where gram amounts of a single-day dose approach the higher range, as optionally for larger weight people, the use of a 3 fluid ounce container for single day dose beverage composition is envisioned to be optionally preferable.

Within the instant invention, a preferred embodiment comprises L-theanine in the range of 100 mg-800 mg, alpha-glycerophosphocholine (alpha-GPC) in the range of 0 mg to 600 mg, L-tyrosine in the range of 50 mg-300 mg, N-acetyl L-tyrosine in the range of 0 mg to 300 mg, and 5-hydroxytryptophan (5-HTP) in the range of milligrams in line with the composition 1 formula listed above. Other ingredients such as B vitamins, at least one flavorant, at least one colorant, and at least one preservative may also be present.

Another preferred embodiment of the instant invention is a composition comprising L-theanine in the range of 100 mg-800 mg, citicoline in the range of 40 mg-600 mg, L-tyrosine in the range of 50 mg-300 mg plus N-acetyl L-tyrosine in the range of 0 mg-300 mg and milligrams of L-tryptophan in line with the composition 2 formula listed above, plus optionally other ingredients, such as B vitamins, malic acid and at least one preservative.

The compositions of the present invention may comprise one or more B vitamins. The vitamin B complex is an ingredient in a preferred embodiment and comprises B1 (thiamine), B2 (riboflavin), B3 (niacin), B5 (pantothenic acid), B6 (pyridoxine), B7 (biotin), B9 (folic acid) and B12 (cyanocobalamin).

Thiamine is a water soluble vitamin and is also known as aneurine and functions as a coenzyme in the oxidative decarboxylation of alpha keto-acids (involved in energy production) and in the transketolase reaction of the pentose phosphate pathway (involved in carbohydrate metabolism). Thiamine is also important in nerve transmission (independently of coenzyme function). As an aside, it is noted that it may also act as an insect repellent.

Riboflavin is a water soluble vitamin and functions as a component of two flavin coenzymes—flavin mononucleotide (FMN) and flavin adenine dinucleotide (FAD). Common forms are riboflavin, riboflavin butyrate and flavin adenine dinucleotide.

Niacin is a water-soluble vitamin and describes the factors that exhibit the biological properties of nicotinamide. It occurs as nicotinamide and nicotinic acid. It is sometimes known as niacinamide. An example of a derivative is benzyl nicotinate. Niacin functions as a component of two coenzymes, nicotinamide adenine dinucleotide (NAD) and nicotinamide adenine dinucleotide diphosphate (NADP). These co-enzymes participate in many metabolic processes including glycolysis, tissue respiration, lipid, amino acid metabolism and purine metabolism.

Pantothenic acid is also a water soluble vitamin and functions mainly as a component of coenzyme A and acyl carrier protein. Vitamin B6 is a water soluble vitamin. Vitamin B6 is a generic term used to describe the factors that exhibit the biological activity of pyridoxine. It occurs in food as pyridoxine, pyridoxal and pyridoxamine. Vitamin B6 is converted in erythrocytes to pyridoxal phosphate and, to a lesser extent, pyridoxamine phosphate. Examples are pyridoxine hydrochloride and pyridoxine di-octanate.

Folic acid (pteroylglutamic acid) is a water soluble vitamin and is the parent factor for a large number of derivatives collectively known as folates. Folate is the generic term used to describe the factors that exhibit the biological activity of folic acid; it is the preferred term for the vitamin present in foods which represents a mixture of related factors (folates).

Vitamin B12 is a water-soluble vitamin and it is the generic term used to describe the factors that exhibit the biological activity of cyanocobalamin. It includes a range of cobalt-containing factors, known as cobalamins. Cyanocobalamin and hydroxocobalamin are the two principal forms in clinical use. One or more of these B vitamins are optionally factors within embodiments of the instant invention edible compositions.

In one or more embodiments, a chelating agent is an ingredient in a preferred embodiment of the instant invention edible compositions, as selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl) ethyleneglycol-N,N,N′,N′-tetraacetic acid (EGTA), trans-1,2-diaminocyclohexane-N,N,N′,N′-tetraacetic acid (CyDTA) or a pharmaceutically acceptable salt thereof (normally as a sodium salt), more preferably EDTA, HEDTA and their salts; most preferably EDTA and its salts.

The compositions of the present invention may optionally comprise other additives. Non-limiting examples are huperzine, L-carnitine and alpha-lipoic acid, in pharmaceutically effective amounts.

The compositions of the present invention may optionally comprise a sweetener. One preferred sweetener is sucralose, typically added in milligrams quantity per single-day dose.

The compositions of the present invention may further comprise stabilizers, antioxidants, antimicrobials, sweetener and buffers and the like, as are known in the art.

The compositions of the present invention may optionally be configured as food look-alikes such as by augmenting standard food production methods, using alterations as are known in the art. For example, compositions can be configured as bits of sweetened candy-like confection for attachment to, or mixing into, common food. This is further discussed below.

The inventive steps utilized to reach these preferred embodiments include understanding and applying the bioavailability response from oral ingestion of the various ingredients, their individual and relative propensity to cross the BBB, and their individual and relative potency for production of the respective neurotransmitter in the brain. Another inventive step involves accounting for the effects of the usage, such as intermittent use or sustained daily use over weeks or months, of the instant invention edible compositions.

As mentioned above, unsafe or excessive serotonin-rich neurotransmitter-precursor-containing compositions are potentially unhealthy when taken daily for months by a mammalian subject, and these are not supported within the instant invention. Such potential for adverse health effect by consumption of unsafe supplements, as earlier discussed, is understood to be more likely for a consuming mammalian subject also eating a nutrient-deficient diet, such as a diet low in protein and/or low in choline and/or a diet deficient in vitamin D. Among the risks are that over-consuming such unsafe compositions may cause the effects of excess serotonin-precursor on brain enzyme activity to become cumulative. Such serotonin-precursor excess can eventually overwhelm the brain's relevant enzymes. Accordingly, the embodiments of the instant invention are comprised such that, when taken by a mammalian subject per instructions provided, these compositions are unlikely to predispose a mammalian subject/consumer to DD or SS.

For the instant invention to provide the above described benefits to mammalian subjects/consumers, it is advantageous for the PR to find the purchase and application of said instant invention compositions to be attractive, effective and convenient. Thus, and as part of the instant invention inventive steps, the choice of mode of providing the instant invention edible composition to PR is analyzed and at least partially optimized. For example, a preferred embodiment can be provided as a liquid suitable for consumption by an animal, such as by squeeze bottle with calibrated scale allowing measurement of dose delivered to pet feeding dish or directly into pet's mouth.

Prior art U.S. Pat. No. 8,187,647 (Bhargava) mentions a mixture of methyl xanthine, choline and other ingredients dispensed in purified water solution of 60 milliliters, i.e. approximately equal to 2 fluid ounces. However, Bhargava's reference and inventive steps differ in fundamental ways from those herein. For example, Bhargava's composition did not comprise a theanine, or BDSP formulation or alpha wave enablement methodology or a tryptophan. Hence the purpose of Bhargava's reference to the 60 milliliter water solution formulation could not have envisaged the herein disclosed invention or its benefits or the associated instructions, as herein disclosed, i.e. to optimize promotion of healthful alpha brain wave activity in the context of relative protection against both SS and DD, while providing improvement in mental function/brain function/activity, such as calming/anxiety relief by improvement in alpha brain wave activity, and providing improvement in brain function, such as improved satiety, thus enabling weight control.

Dispensing of the instant invention edible compositions configured as a beverage, dispensed in a 2 ounce to 3 ounce beverage container, with instructions advising divided dosing and timing, as herein disclosed, enables supplying neurotransmitter-precursors in gradual amounts to a mammalian subject. This assists in avoiding unhealthy spikes in serotonin for the brain, and helps therefore to avoid overwhelming of relevant enzyme activity. Furthermore, dispensing in approximately 2 or 3 fluid ounce container allows a PR to carry a single container of beverage composition which contains the total daily dose needed, then partake of aliquots. The PR can himself/herself sip or alternatively for an animal administer aliquots over the day to conveniently accomplish the benefits of improved mental function, and improved brain function/activity for the specific mammalian subject, then discard container at end of day.

The recommendations/instructions for using/consuming the instant invention edible compositions herein disclosed, are typically listed for a PR's use, for example, on the container label. Additionally or alternatively, usage instruction may be provided to the PR on a website page. Thus, while avoiding the likelihood of DD and/or SS, benefits accrue to the mammalian subject which include the alpha brain wave enablement, an improvement in brain activity, the enhanced satiety, an improvement in brain function, and a boost in mood, as associated with an improvement in the mental status via alpha brain wave enablement. Additional associated benefits can include enhancement of calm focused thought, enabled creativity, and enhanced problem solving and the like.

Thus, the inventive steps in a preferred embodiment include dispensing the instant invention edible composition as a liquid in a container approximating 2 to 3 fluid ounces size, along with providing the relevant usage guidelines as herein disclosed. The small size reclosable container is convenient to carry for the average PR. This ease of use engendered by container size also enables acceptance of, and the following of the guidelines/instructions, the small container being easily carried in pocket or purse, and used during the day unobtrusively. The two fluid ounce or three fluid ounce bottle, as envisioned for some beverage embodiments, as an example, is not required to be refrigerated, due to its beverage's content of stability enhancing ingredients including preservatives, such as sodium benzoate, potassium sorbate, malic acid and EDTA.

In this regard, the preservative ingredients become part of the essence of the invention in such preferred embodiments. The amounts of these ingredients as typically utilized in the above described preferred embodiments are in the range of the following: Sodium benzoate—23 milligrams per 2 fluid ounces, potassium sorbate—23 milligrams per 2 fluid ounces, EDTA—2 milligrams per 2 fluid ounces, malic acid—425 milligrams per 2 fluid ounces.

The benefits of non-refrigeration include the convenience for the PR of carry-away in pocket or purse, and enablement of quick dispensing, for example, where PR opens the container, consumes/administers an aliquot, then recloses the bottle. The PR then can carry the bottle during the day, in pocket or purse, and is able to consume/administer additional aliquots as per instructions. The preservatives in the instant invention render the composition sufficiently resistant to bacterial growth such that little risk obtains for the mammalian subject for PR to open the bottle during the day and consume/administer a first aliquot, close the container, carry it, then reopen the container in 2 hours or as instructed, and consume/administer an additional aliquot, and so on until the container is empty. Other instruction set for some preferred embodiments may include instructions for aliquot consumption at a recommended time relative to a meal or snack or feeding of mammalian subject, and/or optionally for contemporaneous consumption of water by mammalian subject, and/or optionally for an aliquot consumed/administered hourly by the mammalian subject for the 3 hours prior to said mammalian subjects bedtime or the like.

The divided dose dispensing method comports well with allowing the body of the mammalian subject to maintain/replenish its supply of the relevant brain enzymes, and allows less opportunity for the serotonin-precursor ingredients to induce sleepiness because they are subject to ongoing metabolism, therefore with less tendency to deliver a high dose all at once. This alertness preservation occurs because, with the divided dose schedule, these ingredients are only acting at a reduced level, as the body metabolizes earlier sipped aliquot's ingredients, and thus gradually clears from activity in the body those earlier consumed quantities of factors/ingredients.

Furthermore, it is envisioned that busy persons/consumers are habituated to taking with them during the day those convenient articles which they utilize in daily life. Another aspect of the convenience factor of the instant invention edible composition in preferred embodiments of approximately 2 fluid ounce to 3 fluid ounce containers/bottles is that a busy individual is more likely to carry the non-refrigerated container as compared to a cold drink because the cold drink ‘sweats’.

By contrast, some of the instant invention edible composition preferred embodiments may have chemical and biological stability allowing a satisfactorily long shelf life prior to opening and a safety factor of a day or two if re-closed then reopened, during which consumption of aliquots is safe, typically up to 72 hours, after which the beverage should be discarded.

### EXAMPLES

### Example 1

The composition appearing in Table 1 was provided.

TABLE 1
An example of an edible composition single-day dose

| FUNCTION | COMPONENT | AMOUNT (mg) | |
|---|---|---|---|
| An alpha brain | L-Theanine | 100-800 | mg |
| wave- promoting | | | |
| factor | | | |
| A choline provider | alpha- | 40-600 | mg |
| | glycerophosphocholine | | |
| | (alpha-GPC) | | |
| a serotonin-precursor | 5-hydroxytryptophan | 15-270 | mg |
| | (5-HTP) | | |
| A dopamine | L-tyrosine | 50-300 | mg |
| precursor | | | |
| A dopamine | N-acetyl L- | 50-300 | mg |
| precursor | tyrosine | | |
| A dopamine | L-phenylalanine | 50-300 | mg |
| precursor | | | |
| Vitamin B1, | Thiamine | 10-100 | mg |
| thiamine provider | | | |
| Vitamin B6 provider | Pyridoxal 5′-phosphate | 10-100 | mg |
| | (PLP) | | |
| Vitamin B12 | methylcobalamin | 0.1-0.7 | mg |
| provider | | | |
| Folic acid provider | Folic acid | 0.1-4 | mg |
| Preservative | Sodium benzoate | 23 | mg |
| Preservative | potassium sorbate | 23 | mg |
| Buffer or acidity | malic acid | 425 | mg |
| regulator | | | |
| Stability enhancer | EDTA | 2 | mg |
| Total dry weight | | 0.8-3.3 | grams |

| | | |
|---|---|---|
| SOLVENT | WATER | 56.6-85 grams |
| | | (2-3 fluid ounces) |

### Example 2

The composition in Table 2 was provided.

TABLE 2
An example of an edible composition single-day dose

| FUNCTION | COMPONENT | AMOUNT (mg) | |
|---|---|---|---|
| An alpha brain | L-Theanine | 100-800 | mg |
| wave- promoting | | | |
| factor | | | |
| a serotonin-precursor | 5-HTP, from Griffonia | 50-600 | mg |
| | seed extract | | |
| Vitamin B1, | Thiamine | 10-100 | mg |
| thiamine provider | | | |
| Vitamin B6 provider | Pyridoxal 5′-phosphate | 10-100 | mg |
| | (PLP) | | |
| Folic acid provider | Folic acid | 0.1-4 | mg |
| Preservative | Sodium benzoate | 23 | mg |
| Preservative | potassium sorbate | 23 | mg |
| Buffer or acidity | malic acid | 425 | mg |
| regulator | | | |
| Stability enhancer | EDTA | 2 | mg |
| Total dry weight | | 0.6-2.1 | grams |

| | | |
|---|---|---|
| SOLVENT | WATER | 56.6-85 grams |
| | | (2-3 fluid ounces) |

A preferred embodiment of the instant invention includes the use of an edible composition in a weight control method, as further disclosed below. Persons eating excess carbohydrates during meals are known to tend to gain excess weight. Increasing their satiety will allow them to decrease their consumption of carbohydrates and lose weight.

One reason for such weight gain, according to recent research, appears to be the linkage between such excess carbohydrate consumption and the user's sensation of reward or satiety as engendered for example by the neurotransmitter serotonin's production increase and thus its enhanced activity in the user's brain in response to a high-carbohydrate meal. Research (Herrera 2011) has found that consuming carbohydrate-rich meals, especially high-glycemic index meals and high-glycemic load meals, causes increased tryptophan availability to the brain, which thus appears capable of boosting brain serotonin, and thus a reward or satiety sensation.

The mechanism for the increased tryptophan availability to the carbohydrate-eater's brain appears to be that such consumption of carbohydrate elicits a spike in insulin secretion into the blood. The spike of insulin into the blood then enhances peripheral, skeletal muscle uptake of large neutral amino acids from the blood. Not all amino acids circulating in the blood participate in such movement of amino acids from blood into muscle. For example, tryptophan, a serotonin-precursor amino acid circulating in the blood, is bound, apparently by relatively weak forces such as van der Waal's forces (McMeeamy 1958), to albumin in the blood, and therefore tryptophan is somewhat prevented from participating in this insulin-induced movement of amino acids from blood into muscle.

This selective removal of amino acids from the blood under the influence of insulin, leaves tryptophan with temporarily less competition from the blood's other amino acids at the blood brain barrier's amino acid transport mechanism. As a result, such relatively unopposed tryptophan circulating in the blood appears to have an increased opportunity to be taken up by the amino acid transport mechanism at the blood brain barrier (BBB) and thereby increased tryptophan is conveyed into the brain. Without being bound to any one particular theory, it would appear that the increased tryptophan availability in the brain, secondary to such carbohydrate consumption, enables tryptophan to be increasingly enzymatically transformed into serotonin in the brain following such carbohydrate meal.

A mammalian subject, as a result of such an experience of carbohydrate consumption, and in receipt of such rewarding sensation in his/her brain due to such serotonin increase, may thus become habituated to repeat the carbohydrate-eating-to-excess experience over and over. Such unhealthy chronic over-consuming of carbohydrate calories can cause weight gain. To help such individuals break the cycle, a safe and effective timely substitute serotonin production mechanism for satiety within their brains is herein disclosed.

The serotonin substitute satiety-enhancing strategy is provided by beneficial action of consumption of a preferred embodiment of the instant invention edible composition comprising at least one serotonin-precursor factor comprising a tryptophan and at least one alpha brain wave enabling factor comprising theanine. The method of weight control further typically comprises providing for PR, instructions for use of the said edible composition. Such method, in use, induces a relative reduction in mammalian subject's calorie consumption and thereby a weight loss over time.

Through said use of the said composition and method, the brain level of serotonin in the mammalian subject's brain will tend to beneficially increase within several minutes to an hour following consumption of the composition or an aliquot, along with an enablement of alpha brain waves. This occurs as a result of the metabolism within the brain of the serotonin-precursor from the composition, assisted by the action of the theanine in promoting alpha brain waves. Alpha brain waves are known to be associated also with serotonin release/activity in the brain. As previously mentioned, such alpha brain wave activity is optionally further enabled by a choline, as is present in some embodiments of the instant invention edible compositions and/or from diet.

Such serotonin boost provides a substitute sensation to that usually derived from food, i.e. a brain signal of satiety. Such satiety signal, in this case from consuming a low calorie beverage composition of the instant invention rather than from consumption of a high-calorie carbohydrate meal, is beneficial to controlling weight. As a contemporaneous synergistic effect of consumption of the composition, in addition to the serotonin effect, the alpha brain wave enablement may also assist the mammalian user to cut calories. For example, research suggests that alpha brain waves may assist with reducing food cravings (Daubenmier 2011, Lacaille 2014). The induction of alpha brain waves via meditation/mindfulness has been found to be capable of reducing food cravings. Alpha waves are typically also created in doing aerobic exercise, an activity also known to be associated with improved weight control.

The weight control method of applying the instant invention compositions, in some embodiments, includes supply to PR of at least one instruction for use of the edible composition, advising an aliquot of the composition be consumed when mammalian subject feels/expresses/demonstrates hunger, such as a sip when a pet ‘begs’ for food. In a preferred embodiment, the instruction for use comprises timing of consumption of edible composition in relation to pet's usual feeding time or meal time of mammalian subject. A specified aliquot is then typically administered, such as instruction for mammalian subject to consume up to a third of the single-day dose amount of the composition at a time 30 minutes prior to mealtime.

As previously discussed, such timing of consumption of a preferred embodiment for weight control of an edible composition of the present invention enables a timely substitute production of serotonin in the mammalian subject's brain from metabolism of the composition's serotonin-precursor. Such satiety signaling is further and perhaps synergistically enabled by the composition's theanine ingredient's promoting of alpha waves in the subject's brain, and perhaps further enabled by choline action, if is sufficiently present in diet and/or from composition. It is useful to discuss further details of such timing.

Such instructions, optionally including such timing for consumption of a preferred embodiment of instant invention by mammalian subject versus mealtime of mammalian subject, can provide a benefit in ingredient effectiveness for the mammalian subject, such as better absorption opportunity for at least some of the factors comprising the composition. Consuming the instant invention edible composition at a pre-meal moment, when the stomach is typically empty or nearly so, as previously mentioned, enables at least some factors to cross the mucous membrane of the stomach/intestine without excess interference from food. Such interference might otherwise be able to delay or diminish the benefits, and thus discourage the PR/mammalian subject from further use of the compositions. Furthermore, as previously mentioned, with regard to the substitute surge of brain serotonin production timely provided in at least some embodiments of the instant invention by virtue of instructions for timing being followed, the serotonin surge in the brain enables mammalian subject to be satiated on fewer calories.

The amount of composition administered can be tailored to body weight of mammalian subject. For example, it is envisioned that an instant invention edible composition can be dispensed as a dry powder with directions for quarter-teaspoon or half-teaspoon use or the like as a suitable dose depending on body weight of the mammalian subject, as provided for in the instructions. Typically such dry powder is consumed only after first being dissolved in water.

Thus, by repeated use of the compositions of the present invention, in a timely fashion over days and weeks, a mammalian subject is provided substitute serotonin surges within the context of alpha brain wave enablement, which combination at least partially pre-empts excess carbohydrate consumption. This assists the mammalian subject to gradually reduce weight to more healthful range, reducing his/her risks of High-Body-Mass-Index-associated diseases, such as, but not limited to, diabetes, cardiovascular disease, stroke and many others.

One embodiment for a weight control method's edible composition of the instant invention is a lozenge suitable for under the tongue application/absorption of ingredients. For example, such a composition comprises 200 mg of theanine and 100 mg of 5-HTP. Another embodiment for the weight control method's edible composition is a gummy chewable condiment, comprising for example 250 mg of theanine and 300 mg of tryptophan. The timing of consumption at specified minutes prior to next meal/snack, as optionally provided for in the instructions, can vary in minutes prior, depending on the chemical nature of the serotonin-precursor and absorbability across oral mucosa.

The timing may also be different as determined by absorption characteristics relative to the carrier used. For example, a 5-HTP fast-dissolving, beneath-the-tongue, absorbable lozenge will typically have a lower number of minutes pre-meal as instruction suitable for pre-meal/pre-snack application. For example, the instruction may be to consume the lozenge, 0-10 minutes, 10-20 minutes, 20-30 minutes, or 30 minutes to one hour before the next meal/snack.

By comparison, and in accordance to another embodiment of the present invention, a slower acting/slower digestible chewable gummy condiment with tryptophan as the serotonin-precursor, rather than 5-HTP, typically the instructions for use will have a greater number of minutes for pre-meal/pre-snack application, such as instruction to administer/consume the gummy condiment 60-80 minutes, 80-100 minutes, 100-120 minutes, or 2-3 hours before the next meal or snack.

According to a further embodiment of the present invention, it is further envisioned that instructions for use contain instruction for mammalian subject to consume water, typically one or two 8 ounce glasses full of water, to be consumed at or near the time of consumption of composition. Such water consumption tends to fill stomach at least partially and serves at least a dual function of reducing chance for nausea from composition ingredients while augmenting weight loss (Parretti, 2015).

According to a further embodiment of the present invention, it is further envisioned that the strength of the serotonin-precursor amounts in the said edible compositions can be sequentially reduced in subsequent edible compositions to be consumed by mammalian subject over time. This graduated-over-time reduction in potency of the edible compositions serves to still engender a pharmacologically active serotonin surge in mammalian subject's brain while avoiding SS, DD, and avoiding habituation of the mammalian subject in an unhealthy way to the serotonin reward.

As an optional addition to the said timed instructions, it is envisioned to also advise consumption of coffee by a mammalian subject at or near the time of consumption of the said composition. Consuming coffee provides caffeine to the brain. Caffeine in the brain exerts an antagonist action at the glycine receptor (GlyR) of neurons (Beecham 2015), thereby reducing chloride inflow into the neuron, thus presumably reducing polarity of neuron membranes. The effect of caffeine within the brain has the capacity to enable the brain to a more alert and focused mode, especially when theanine is also active within the brain (Owen 2008), thus counteracting any tendency to drowsiness of theanine action from the composition. An alert focused mind is an added benefit to a person, such as during daytime, when alertness and focus is useful in enabling completion of daily tasks.

A sweetener is an optional additional ingredient. Also envisioned is a low calorie sweetener, such as sucralose, as an optional ingredient within the instant invention edible compositions. A variety of low calorie condiments and candies of instant invention composition are envisioned, such as, without limitation, a hard candy, gum drop, jelly bean, chocolate confection, chewing gum, chewable mint, taffy-like candy, soft-center candy, wafer, sprinkle applicable onto food, common food look-alikes. These may be prepared using well-known methods, with addition of the composition ingredients.

For edible compositions of the instant invention usefully configured as common food look-alikes, it is useful to restrict children's access to such products, such as by keeping the products under lock and key or the like. This restriction will preclude the tendency for children to overeat such compositions.

For example, some edible compositions of the instant invention configured as food look-alike are envisioned to include, without limitation, an ice cream confection, a popsicle, a cookie, a cake, a crunchy snack such as a cracker or chip, a spread, a cheese, a milk, a yogurt or the like. One method to prepare such common food look-alikes is by sprinkling onto a previously baked or prepared product a helping of sprinkle candy bits comprising the active factors/ingredients of a composition in accordance to the instant invention.

Alternatively, the bits can be provided in a separate container whereby the PR can open that container and drop the bits onto the food, optionally mixing them into the food, prior to the food's consumption. When pre-applied during manufacture, such sprinkle bits can be made to adhere to the food item, for example via use of a standard food grade binder or the like. Alternatively, when pre-applied to products such as milk, cheese, yogurt, spreads or the like, the sprinkles can be mixed into the product during production, or alternately applied as a layer on the surface of such products prior to the product being capped or container closed. Labels typically list the amounts of the factors, and a caution not to over-consume or allow children access to these products.

It is envisioned that where mammalian subjects are likely to be consuming multiple types of products containing compositions of the instant invention, especially food look-alikes, instructions for use can include instructions to limit such consumption, with consideration of the total amount of such factors consumed in a day, and details of how to calculate relevant neurotransmitter-precursor amounts to keep consumed amounts of these factors within a safe range.

A weight control edible composition herein disclosed is optionally envisioned to be dispensed in the form and manner of a beverage, as previously mentioned, optionally within a graduated container, and optionally secured from inadvertent access by children, such as by a child-safe cap. Furthermore, a weight control edible composition of the instant invention is optionally envisioned to be incorporated into a kit, similar to that described hereinbefore, with instructions supplied for use by PR. A preferred embodiment of the instant invention is configured as animal food, such as pet food. For example, to reduce obesity in a family pet or farm animal or and the like, a preferred embodiment of the instant invention edible compositions comprises a composition containing the factors hereinabove described, mixed into chow, and instructions for use which indicate how much chow to feed to a pet dog of a certain weight, or the like.

An animal thus routinely or intermittently satiated via consuming such compositions, such as several days per week of the like, may tend to ‘beg’ less for food. A reduction in the animal's ‘begging’ relieves PR guilt, and thus provides PR increased motivation for reducing the amount of food he or she feeds to the animal, thus tending to bring animal's weight into more healthful range.

To further clarify the specifics of preferred embodiments for enabling satiety for a human adult, for a human adolescent or for a pet dog, reference is made to the earlier disclosed example in Table 1 above (page 35 herein). For a 300 pound overweight adult human, the higher range of the milligram amounts of the listed factors in composition disclosed in Table 1 might be approximate in a single-day dose, typically delivered in timed aliquots. By constrast, for a 150 pound obese adolescent human, the mid-range milligram amounts of the listed factors of the composition disclosed in Table 1 might be approximate as a single-day dose, again typically delivered in timed aliquots. For an obese pet dog of 40 pounds weight, the lower milligram amounts of the listed factors in the composition disclosed in Table 1 might be approximate, again typically delivered in timed aliquots.

Pet food embodiments of the instant invention edible compositions optionally include digestible snacks and standard pet food as look-alikes. Such pet food compositions are typically accompanied by instructions for PR whereby the consumption dose is configured for adjustment according to animal's weight, such as for pet cats and pet dogs, versus the typical weights of healthy pets and breeds by body length or the like.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

The references cited herein teach many principles that are applicable to the present invention. Therefore the full contents of these publications are incorporated by reference herein where appropriate for teachings of additional or alternative details, features and/or technical background. It is to be understood that the invention is not limited in its application to the details set forth in the description contained herein. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

1. An edible composition for improving at least one health-related characteristic in a mammalian subject, the composition comprising:
a) at least one alpha brain wave promoting factor comprising theanine; and
b) at least one neurotransmitter-precursor comprising factor.

2. An edible composition according to claim 1, further comprising a preservative and configured as a beverage.

3. An edible composition according to claim 1 further comprising at least one choline providing factor.

4. An edible composition according to claim 1, configured and dispensed as a dry powder.

5. An edible composition according to claim 4, wherein the neurotransmitter-precursor comprising factor comprises an extract of Griffonia simplicifolia seeds.

6. An edible composition according to claim 2, wherein the neurotransmitter-precursor comprising factor comprises an extract of Griffonia simplicifolia seeds.

7. An edible composition according to claim 6, wherein the neurotransmitter-precursor comprising factor further comprises at least one dopamine-precursor and at least one serotonin-precursor, and wherein the composition's serotonin-precursor is in metabolically relative amounts to its dopamine-precursor so as to at least partially promote, in use, healthful balance between serotonin production and dopamine production within the brain of said mammalian subject.

8. An edible composition according to claim 5, wherein the neurotransmitter-precursor comprising factor further comprises at least one dopamine-precursor and at least one serotonin-precursor, and wherein the composition's serotonin-precursor is in metabolically relative amounts to its dopamine-precursor so as to at least partially promote, in use, healthful balance between serotonin production and dopamine production within the brain of said mammalian subject.

9. An edible composition according to claim 2, wherein the neurotransmitter-precursor comprising factor comprises a serotonin-precursor selected from 5-hydroxytryptophan (5-HTP), L-5-HTP, a hydroxytryptophan, L-tryptophan, an extract of Griffonia seeds, a substantially purified tryptophan, a tryptophan, a tryptophan derivative and combinations thereof.

10. An edible composition according to claim 4, wherein the neurotransmitter-precursor comprising factor comprises a serotonin-precursor selected from 5-hydroxytryptophan (5-HTP), L-5-HTP, a hydroxytryptophan, L-tryptophan, an extract of Griffonia seeds, a substantially purified tryptophan, a tryptophan, a tryptophan derivative and combinations thereof.

11. An edible composition according to claim 2, comprising in single-day dose at least one of: a hydroxytryptophan in amount greater than 15 milligrams and less than 1801 milligrams, a tryptophan in amount less than 6 grams.

12. An edible composition according to claim 4, comprising in single-day dose at least one of: a hydroxytryptophan in amount greater than 15 milligrams and less than 1801 milligrams, a tryptophan in amount less than 6 grams.

13. An edible composition according to claim 1, wherein the neurotransmitter-precursor comprising factor comprises 5-hydroxytryptophan in pharmaceutically effective amount and wherein the composition is dispensed configured in at least one of a pill, a lozenge, a sublingual, a tablet, a capsule, a dry powder, a liquid, a solid, a semi-solid, a solution, a suspension, a gel, a gelatin, a hard candy, a soft candy, a condiment, a gum drop, a jelly bean, a chocolate confection, a chewing gum, a mint, a taffy-like candy, a soft-center candy, a wafer, a sprinkle applicable onto food, a fizzy effervescent drink tablet, a spray, a wash, a beverage, a common food look-alike, animal food, and combinations thereof.

14. An edible composition according to claim 2, wherein the neurotransmitter-precursor comprising factor comprises a plant extract containing 5-HTP and wherein the beverage comprises a pharmaceutical aqueous carrier comprising 50-88 ml water.

15. An edible composition according to claim 1, comprising in single-day dose a tyrosine, a theanine, 20 to 1800 mg and wherein the neurotransmitter-precursor comprising factor comprises a 5-hydroxytryptophan-comprising factor (5-HTP) wherein the 5-HTP content is within the range of 15-1800 mg.

16. An edible composition for weight control comprising in single-day dose a theanine, and a 5-hydroxytryptophan (5-HTP) comprising factor wherein the 5-HTP content is within the range of 15-1800 mg.

17. A method for improving brain function/activity in a mammalian subject, the method comprising providing the subject with at least one composition according to claim 16, in a pharmaceutically effective amount, thereby in use improving brain function/activity in said mammalian subject.

18. A method according to claim 17, wherein said at least one composition further comprises said composition in calibrated volume container, and wherein instructions for consumption are provided.

19. A method according to claim 18, wherein said instructions comprise at least one instruction regarding consumption of a volume dose of said composition within a predetermined period of time.

20. A method according to claim 17, wherein said improving of brain function/activity relates at least partially to satiety.

21. A method according to claim 17, wherein said improving of brain function/activity relates at least partially to weight loss.

22. A method according to claim 18, wherein said instructions further comprise at least one instruction regarding said subject consuming the contents of more than one calibrated volume-dose container per day.

23. A method for enabling contemporaneous improvement in mood and reduction in food hunger in a mammalian subject, said method comprising providing said subject at least one consumable comprising:
a) a theanine in pharmaceutically active amount; and
b) at least one neurotransmitter-precursor in pharmaceutically active amount.

24. A kit for improving brain function/activity in a mammalian subject, the kit comprising:
a) an edible composition according to claim 2;
b) a calibrated container of up to 4 fluid ounces capacity for containing said edible composition; and
c) instructions for use of the kit, wherein the composition comprises sufficient preservative so as to preclude a requirement for refrigeration of said composition in common use.

25. A method of improving brain function/activity in a mammalian subject, the method comprising providing a series of edible compositions, each as defined in claim 1, to said mammalian subject, and wherein an amount of at least one neurotransmitter-precursor within a first composition of said series differs from an amount of said at least one neurotransmitter-precursor in a second composition of the series.

26. A method for improving weight control for a mammalian subject, the method comprising providing said mammalian subject with at least one edible composition comprising at least one serotonin-precursor containing factor and at least one theanine containing factor.

27. The method of claim 26, wherein said at least one serotonin-precursor containing factor comprises an extract of Griffonia simplicifolia seeds.

28. The method of claim 27, further comprising providing instructions for use of said at least one edible composition, wherein said instructions comprise at least one element regarding timing of consumption of at least a portion of said composition.

29. The method of claim 28, wherein the composition is provided in a form selected from the group consisting of a pill, a lozenge, a sublingual, a tablet, a capsule, a dry powder, a liquid, a solid, a purified water solution, a suspension, a hard candy, a soft candy, a condiment, a gum drop, a jelly bean, a chocolate confection, a chewing gum, a mint, a taffy-like candy, a soft-center candy, a wafer, a sprinkle applicable onto food, a fizzy effervescent drink tablet, a spray, a wash, a beverage, a common food look-alike, animal food, and combinations thereof.

30. The method of claim 27 further comprising use of a kit, the kit comprising:
a) said edible composition at least partially configured as a beverage;
b) a calibrated container of up to 4 fluid ounces capacity for containing said beverage; and
c) instructions for use of the kit, wherein the said beverage comprises sufficient preservative so as to preclude a requirement for refrigeration of said beverage in common use.

31. A method for reducing a likelihood of reflux of stomach acid into the esophagus of a mammalian subject, said method comprising providing said subject with at least one consumable comprising:
a) a theanine in pharmaceutically effective amount; and
b) at least one neurotransmitter-precursor in pharmaceutically effective amount.

32. The method of claim 31 further comprising providing instruction to said subject to consume said consumable within a timeframe, wherein said timeframe is related to time said subject goes to sleep.
